(19) United States
(12) Patent Application Publication
(10) Pub. No.: US 2016/0335770 A1
(43) Pub. Date: Nov. 17, 2016

(54) SYSTEM AND METHOD FOR THREE-DIMENSIONAL QUANTITATIVE EVALUAITON OF UTERINE FIBROIDS

(71) Applicant: KONINKLIJKE PHILIPS N.V., Eindhoven (NL)

(72) Inventors: MING DE LIN, MIDLE RIVER, MD (US); JULIUS CHAPIRO, BERLIN (DE); KELVIN KEIVIN HONG, WOODSTOCK, MD (US)

(21) Appl. No.: 15/111,850

(22) PCT Filed: Jan. 19, 2015
(86) PCT No.: PCT/IB2015/050379

§ 371 Date: Jul. 15, 2016

Related U.S. Application Data
(60) Provisional application No. 62/022,695, filed on Jul. 10, 2014.
(60) Provisional application No. 61/930,989, filed on Jan. 24, 2014.

(51) Int. Cl. G06T 7/00; A61B 6/03; A61B 5/026; A61B 6/00

(52) U.S. Cl. CPC G06T 7/0014; A61B 6/481; A61B 6/4085; A61B 6/03; A61B 6/486; A61B 6/507; A61B 5/0263; A61B 6/5217; G06T 7/0081; G06T 2207/10096; G06T 2207/30004; G06T 2207/10028

(57) ABSTRACT

A system (100) and method which assess the results of uterine fibroid embolization. A fibroid lesion is three-dimensionally segmented by a segmentation module (110) on contrast-enhanced arterial phase MRI or CBCT images and a differentiation process performed by a subtraction module (114) identifies actual contrast enhancement from background enhancement. The three-dimensional segmentation mask is then applied to the differentiated image and a region of interest is defined by a comparison module (116) in order to define a normalized threshold. A computation module (112) performs a voxel-by-voxel analysis of enhancing fibroid volume and quantifies the viable enhanced fibroid volume and overall fibroid volume in absolute numerical figures.

### RELATED U.S. APPLICATION

This application claims the benefit of U.S. Patent Application Ser. No. 61/930,989 filed on Jan. 24, 2014 and 62/022,695, filed on Jul. 10, 2014.

This invention was made with government support under grant no. R01 CA160771-01 awarded by the National Cancer Institute of the United States National Institutes of Health. The government has certain rights in the invention.

### BACKGROUND

1. Technical Field

This disclosure relates to image guided intervention, therapy and evaluation, in particular, to an apparatus and method for obtaining reproducible, three-dimensional quantitative assessment of uterine fibroids before, during and after inter-arterial therapy.

2. Description of the Related Art

Uterine fibroids are the most common benign tumors in females, typically diagnosed in the middle reproductive years. Clinically, uterine fibroids can lead to heavy menstrual bleeding as well as bulk-related symptoms such as severe lower abdominal pain and in some patients even infertility. Most patients require surgical treatment which oftentimes leads to permanent sterility.

The role of uterine artery embolization (“UAE”) has evolved as a well-accepted, minimally-invasive alternative to surgical treatment in the management of uterine fibroids. This catheter-based procedure causes irreversible ischemic injury to uterine fibroids while maintaining perfusion of the healthy uterus, which is known to return to normal within 4 months after treatment.

While magnetic resonance imaging (“MRI”) is considered to be the most accurate imaging technique to assess UAE effects, evaluation of treatment response to UAE relies on individual anatomic measurements of fibroid volume by using the formula for a prolate ellipse. In addition, visual assessment of contrast enhancement on follow-up images serves as a measure of fibroid viability. These methods rely on the assumption that fibroid growth or response to UAE occurs in a symmetrical, spherical manner and can be reliably measured by subjective, visual assessment. However, these techniques lead to reader bias and imprecision. In addition to lacking reliability, the measurements are difficult to reproduce.

Numerous studies have failed to achieve a reliable correlation between imaging results and clinical symptom relief. Accordingly, the studies have failed to establish contrast-enhanced MRI as a suitable tool for evaluating fibroid response to UAE or to detect insufficiently treated patients. These circumstances underline the need to create instruments that are capable of accurately quantifying the viable tissue within fibroids on intra- and post-procedural imaging.

From a pathological stand point, uterine fibroid response to UAE happens gradually. While fibroid death is known to occur within 72 hours after embolization, lesion shrinkage may follow over a period of several weeks. These changes can be observed on a contrast-enhanced MRI immediately after the procedure as well as on late follow-up scans (4-12 months after treatment). Accordingly, reporting enhancement in relative terms such as by a percentage of the entire lesion may only be accurate for MRIs taken early after the UAE procedure with the constraint that only minor clinical symptom relief may be expected as early as 72 hours after the treatment. The correlation of late follow-up MRI results with clinical symptoms using a relative approach may be inaccurate.

### SUMMARY

In accordance with the present principles, a system for three-dimensional quantitative evaluation includes a segmentation module configured to perform a three-dimensional segmentation on a contrast-enhanced imaging of a target and to compute the total volume based on the three-dimensional segmentation. The system also includes a subtraction module configured to differentiate actual contrast enhancements from background or baseline enhancements on the contrast-enhanced imaging and to apply a three-dimensional segmentation mask obtained from the three-dimensional segmentation on the differentiated actual contrast enhancements. A comparison module is configured to define a non-enhanced target portion as a region of interest and to compute contrast statistics for the region of interest in order to determine a normalized threshold value for target enhancement. A computation module is configured to define the enhanced target as voxels within the three-dimensional mask based on the threshold value and quantifying the volume of the enhanced target.

In another embodiment, a system for three-dimensional quantitative evaluation includes a processor, an imaging system comprising magnetic resonance imaging or cone beam computed tomography, and a segmentation module configured to perform a three-dimensional segmentation on a contrast-enhanced imaging of a dominant lesion of the uterine fibroids and to compute the total lesion volume based on the three-dimensional segmentation. A subtraction module is configured to differentiate actual contrast enhancements from background or baseline enhancements on the contrast-enhanced imaging and to apply a three-dimensional segmentation mask obtained from the three-dimensional segmentation on the differentiated actual contrast enhancements. A comparison module is configured to define a non-enhanced tissue portion as a region of interest and to compute contrast statistics for the region of interest in order to determine a normalized threshold value for tissue enhancement. A computation module is configured to define the enhanced fibroid tissue as voxels within the three-dimensional mask based on the threshold value and quantify the volume of the enhanced fibroid tissue.

In another embodiment, a method for three-dimensional quantitative evaluation of uterine fibroids includes the steps of performing a three-dimensional segmentation on a contrast-enhanced imaging of a target and computing total target volume based on the three-dimensional segmentation, differentiating actual contrast enhancements from background or baseline enhancements on the contrast-enhanced imaging, applying a three-dimensional segmentation mask obtained from the three-dimensional segmentation on the differentiated actual contrast enhancements, defining a non-enhanced target portion as a region of interest and computing contrast statistics for the region of interest in order to determine a normalized threshold value for target enhancement and defining the enhanced fibroid tissue as voxels within the three-dimensional mask based on the threshold value and quantify the volume of the enhanced fibroid tissue.

These and other objects, features and advantages of the present disclosure will become apparent from the following detailed description of illustrative embodiments thereof, which is to be read in connection with the accompanying drawings.

### BRIEF DESCRIPTION OF DRAWINGS

This disclosure will present in detail the following description of preferred embodiments with reference to the following figures wherein:

FIG. 1 is a block/flow diagram showing a system which employs three-dimensional quantitative evaluation of uterine fibroids in accordance with one illustrative embodiment;

FIG. 2 is a block/flow diagram showing a method for three-dimensional quantitative evaluation of uterine fibroids in accordance with one illustrative embodiment;

FIG. 3 shows images of a lesion treated in accordance with the present principles;

FIG. 4 illustrates a relative versus absolute quantification of lesion enhancement in scenarios without fibroid shrinkage and with fibroid shrinkage; and

FIG. 5 is an illustrative example of differentiation performed by image subtraction in accordance with the present principles.

### DETAILED DESCRIPTION OF EMBODIMENTS

In accordance with the present principles, a three-dimensional quantitative evaluation of uterine fibroids is performed in which absolute quantification of the overall fibroid volume and the enhancing fibroid volume is performed. The system and method provide a reproducible and accurate quantification of the viable total fibroid volume and enhancing fibroid volume in order to measure uterine fibroid enhancement.

The system performs a three-dimensional segmentation of a fibroid lesion on contrast-enhanced arterial phase MRI or cone-beam computed tomography (“CBCT”) images. The total volume of the lesion is computed from the three-dimensional segmentation. A differentiation process is then employed which distinguishes between actual contrast enhancement and background or baseline enhancement. Background or baseline enhancements include false positive enhancements in the image as well as noise. The three-dimensional segmentation mask is then applied to the differentiated image containing the actual contrast enhancement.

A region of interest (“ROI”) is defined on non-enhancing soft tissue in the image and a normalized threshold for tissue enhancement is computed based on the ROI. A voxel-by-voxel analysis of enhancing fibroid volume is then performed in order to quantify the viable fibroid volume in absolute numerical figures. A color map overlay normalized to the maximum intensity in the magnetic resonance (“MR”) image per patient may then be used to demonstrate the distribution and intensity of the enhancement.

The system and method provide a reliable, reproducible and accurate quantification of (i) the overall fibroid volume and (ii) the enhancing fibroid volume in absolute units such as cm³ in order to assess fibroid viability. The system and method for the assessment of uterine fibroids provides precise intra-procedural feedback using CBCT and increases the diagnostic performance of contrast-enhanced MRI. The system and method can create, among other advantageous features, an accurate, reproducible and reliable model to assess the efficacy of UAE.

It should be understood that the present invention will be described in terms of medical imaging. However, the teachings of the present invention are much broader and in some embodiments, the present principles are employed in quantitatively evaluating complex biological or mechanical systems. In particular, the present principles are applicable to internal evaluation procedures of biological systems, procedures in all areas of the body such as the lungs, liver, brain, uterus, gastro-intestinal tract, excretory organs, blood vessels, and any other solid organ tissue, tumor tissue and homogenously or heterogeneously enhancing structures of the body. The elements depicted in the Figs. may be implemented in various combinations of hardware and software and provide functions which may be combined in a single element or multiple elements.

The functions of the various elements shown in the Figs. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Similarly, it will be appreciated that various processes may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W), Blu-Ray™ and DVD.

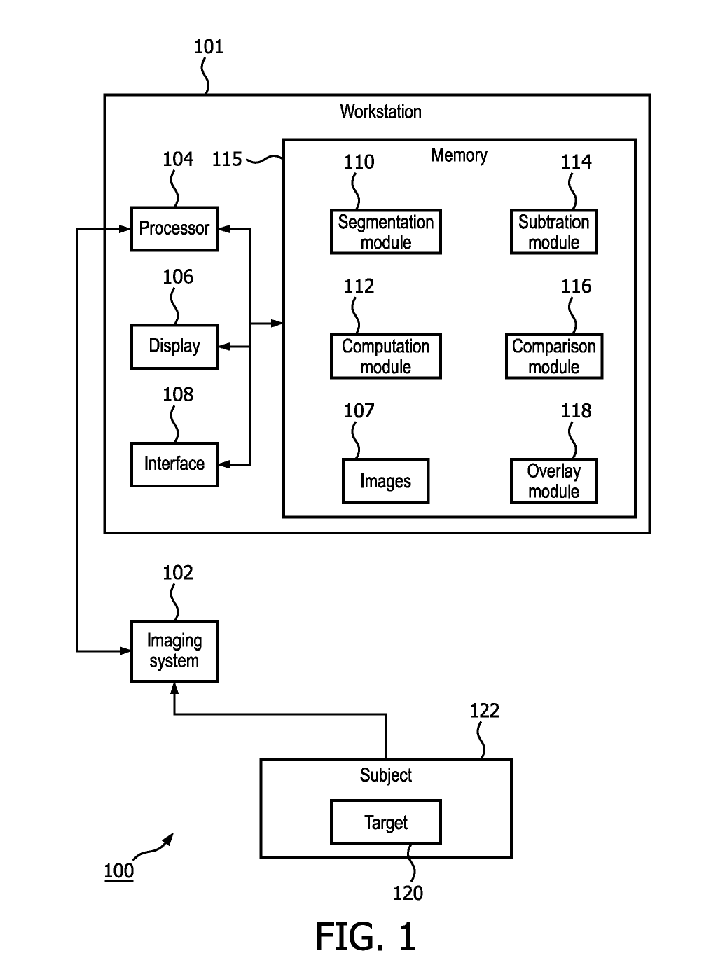

In accordance with the present principles, a three-dimensional quantitative evaluation of uterine fibroids is performed in order to accurately evaluate uterine fibroid enhancement in absolute numeric values. Referring now to the drawings in which like numerals represent the same or similar elements and initially to FIG. 1, a block diagram shows a system 100 constructed in accordance with the present principles. The system 100 may include a workstation 101 from which a procedure is supervised and/or managed. The workstation 101 preferably includes one or more processors 104, memory 115 for storing programs and applications and a display 106 which permits a user to view images and interact with the workstation 101. The system 100 may further include an interface 108 which may include a keyboard, mouse, a joystick, a haptic device, or any other peripheral or control to permit user feedback from and interaction with the workstation 101.

The system 100 includes an imaging system 102 which generates images 107 of a target 120 of a subject 122, such as a uterine fibroid of a female patient. The imaging system 102 may be arterial-phase MRI or arterial-phase CBCT such as to provide intra-procedure feedback. In alternative embodiments, the imaging system 102 may include computed tomography, ultrasound or other imaging systems known in the art. The imaging system 102 may be a separate unit from the workstation 101.

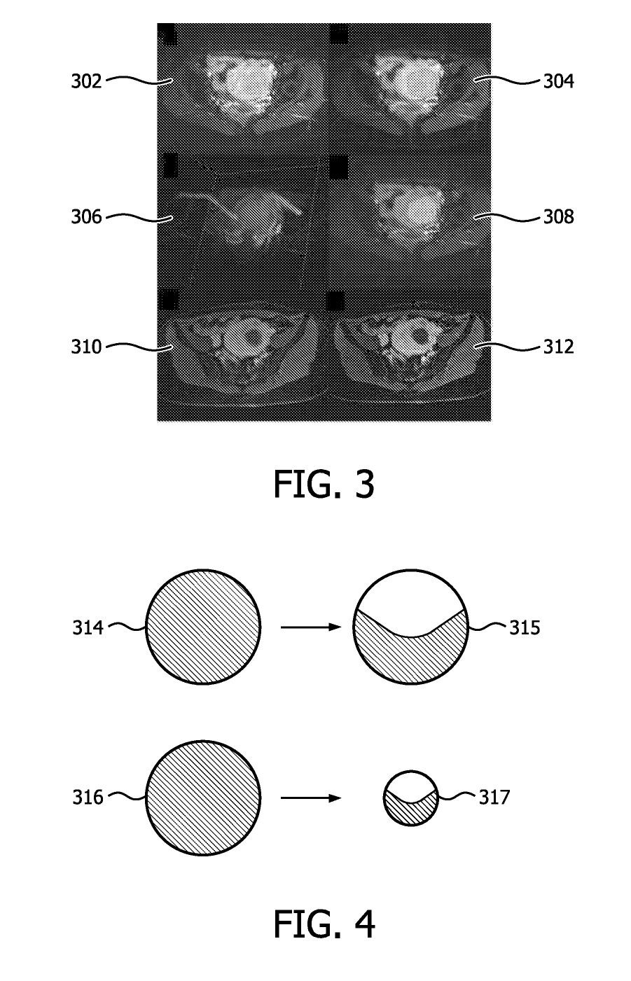

In one embodiment, the imaging system 102 is configured to provide contrast-enhanced MR images of the fibroid lesion. Image 302 of FIG. 3 shows a representative, contrast-enhanced baseline MRI scan.

The system 100 includes a segmentation module 110 which performs a three-dimensional segmentation of the contrast-enhanced MR images of the fibroid lesion. In one embodiment, a semi-automatic three-dimensional tumor segmentation is performed using a software program such as a software prototype (MEDISYS™, Philips Research, Suresnes, France). The software program may be stored in the memory 116 and configured to be accessed by the segmentation module 110. Alternatively, the software program may be stored on a non-transitory computer readable medium which is accessed by the segmentation module 110 and executed by the processor 104. The software may use non-Euclidean radial basis functions in order to perform the segmentation.

Image 304 of FIG. 3 shows the three-dimensional tumor segmentation as performed by the segmentation module 110. The segmentation that was performed in image 304 of FIG. 3 includes the entire lesion. Image 306 of FIG. 3 shows the volume rendering for the segmented lesion in a maximum intensity projection.

The system 100 includes a computation module 112 which computes the volume of the entire lesion based on an analysis of the voxels in the three-dimensional segmentation. However, in alternative embodiments, the computation may be performed by the segmentation module 110.

A resulting three-dimensional segmentation mask is obtained from the segmentation module 110. The segmentation mask is used for the quantitative evaluation of fibroid response to UAE on the contrast-enhanced arterial-phase MRI or arterial-phase CBCT. In order to accurately evaluate the fibroid response to UAE, the system 100 is configured to differentiate between background enhancements and the actual contrast enhancement of the fibroid lesion.

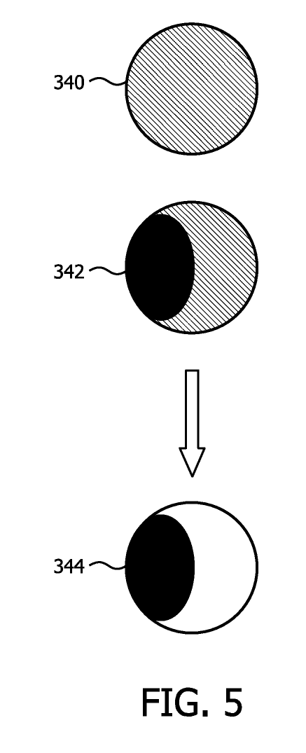

In one embodiment, the differentiation is performed by image subtraction. In this embodiment, a pre-contrast scan, such as an MRI scan, and the arterial-phase contrast-enhanced scan are provided to a subtraction module 114. Image 340 in FIG. 5 shows an illustrative example of a pre-contrast image that is provided to the subtraction module 114. The enhancement shown in image 340 is comprised entirely of background or baseline enhancement. Image 342 in FIG. 5 shows an illustrative example of the contrast-enhanced scan that is provided to the subtraction module 114. Image 342 contains actual enhancement as well as baseline enhancement. The subtraction module 114 is configured to subtract the arterial-phase contrast-enhanced MRI scan in order to remove background enhancement and generate a subtraction image.

The system then transfers the three-dimensional segmentation mask to the actual contrast enhancement. For instance, when the differentiation is performed by image subtraction, the subtraction module 114 receives the three-dimensional segmentation mask from the segmentation module 110, transfers the mask to the subtraction image and generates a clarified image displaying the actual contrast enhancement image with baseline enhancement removed. Image 344 in FIG. 5 shows the subtraction image that is prepared by the subtraction module having the baseline enhancement removed.

The system 100 includes a comparison module 116 that defines a region of interest on non-enhancing tissue, such as soft tissue, in the image in order to compute a normalized threshold for tissue enhancement. The ROI may be, for example, the left psoas muscle in the images shown in FIG. 3.

In accordance with the present principles, the comparison module 116 defines enhanced fibroid tissue as voxels within the three-dimensional mask based on a threshold value wherein the enhancement exceeds the average plus two times the standard deviation value of the ROI. The enhanced fibroid tissue may be defined by a different threshold value as appropriate. In order to estimate fibroid infarction, non-enhancing areas are assumed to be largely necrotic. The volume of enhancing portions is then quantified by the computation module 112 based on an analysis of the number of voxels and expressed in cm³ as well as a percentage of the previously calculated overall fibroid volume.

The system may be further configured to provide a color map overlay normalized to the maximum intensity in the MRI per patient in order to demonstrate the distribution and intensity of the enhancement. The color map overlay may be provided by an overlay module 118. For example, image 308 in FIG. 3 is an image of the lesion shown in images 302, 304, 306 in FIG. 3 with the color map overlay to demonstrate the distribution and intensity of the enhancement. The color red represents maximum enhancement and the color blue represents no enhancement. Image 310 of FIG. 3 shows contrast-enhanced follow-up MRI scan from the same patient. Image 312 of FIG. 3 shows the follow-up MRI scan from the same patient with the color map overlay.

The system 100 is configured to send the images and the rendered computations to the display 106 for viewing by the user. The system 100 may also have output means in order to print the results or the system 100 may electronically send the images and computations over a network.

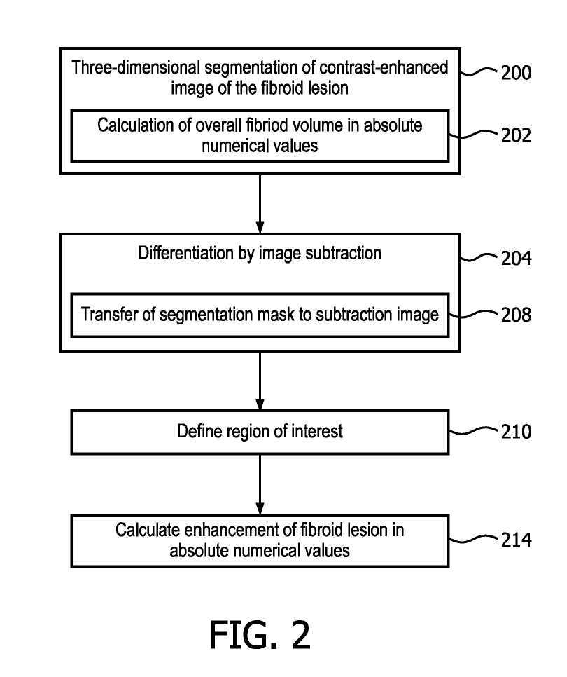

Referring to FIG. 2, methods for three-dimensional quantitative evaluation of uterine fibroids are illustratively shown in accordance with the present principles. In block 200, a three-dimensional segmentation of contrast-enhanced images of the fibroid lesion is performed. The images may be arterial-phase MRI, MRI, CBCT, computed tomography, or ultrasound images. The semi-automatic three-dimensional tumor segmentation is performed using a software program such as a software prototype (MEDISYS™, Philips Research, Suresnes, France) which is run on a suitable computer processor. The software may use non-Euclidean radial basis functions in order to perform the segmentation. In block 202, computation of the overall fibroid volume is performed based on an analysis of the voxels in the three-dimensional segmentation.

In block 204, a differentiation step is performed in order to distinguish between background or baseline enhancements and the actual contrast enhancement of the fibroid lesion. In the embodiment shown in FIG. 2, the differentiation is performed by image subtraction. In this embodiment, the pre-contrast MRI scan is subtracted from the arterial-phase contrast enhanced MRI scan in order to remove background enhancement.

In block 208, the three-dimensional segmentation mask is transferred to the actual contrast enhancement provided by the differentiation step. For instance, when the differentiation is performed by image subtraction, the three-dimensional segmentation mask is transferred to the subtraction image and a clarified image is generated displaying the actual contrast enhancement image with background or baseline enhancement removed. In block 210, a region of interest is defined on non-enhancing tissue, such as soft tissue, in the clarified image. A normalized threshold for tissue enhancement is computed. The ROI may be, for example, the left psoas muscle in the images shown in FIG. 3.

In block 214, enhanced fibroid tissue is computed in absolute numerical figures by defining enhanced fibroid tissue as voxels within the three-dimensional mask based on a threshold value wherein the enhancement exceeds the average plus two times the standard deviation value of the ROI. The enhanced fibroid tissue may be defined by a different threshold value as appropriate. In order to estimate fibroid infarction, non-enhancing areas are assumed to be largely necrotic. The volume of enhancing portions is then quantified based on an analysis of the number of voxels and expressed in cm³ as well as a percentage of the previously calculated overall fibroid volume.

In other embodiments, a color map overlay normalized to the maximum intensity in the MRI per patient may be used to demonstrate the distribution and intensity of the enhancement.

While the method is shown in FIG. 2, with respect to a plurality of steps, one or more steps may be eliminated and still fall within the principles of the method.

The system and method provide absolute numerical figures for the volume of the viable enhanced fibroid as well as the overall fibroid volume in order to assess the levels of fibroid viability and determine the fibroid response to UAE. The quantification of overall fibroid volume and enhancing fibroid volume in absolute numeric values provides a more accurate and reproducible indication of uterine fibroid enhancement. For example, FIG. 4 displays a comparison of the evaluation of lesion enhancement in relative terms versus absolute numerical values. Image 314 of FIG. 4 shows a uterine fibroid lesion prior to UAE wherein there is 100% enhancement and the enhancing volume is 100 cm³. Image 315 of FIG. 4 shows a uterine fibroid lesion after UAE in a scenario without fibroid shrinkage. In image 315 of FIG. 4, there is 50% enhancement and the enhancing volume is 50 cm³. This scenario is commonly seen in a patient during the first few days after UAE is performed. In images 314 and 315 of FIG. 4, the measurement of fibroid lesion enhancement is identical when measured in both absolute and relative values.

In contrast, in images 316 and 317 of FIG. 4, images of a uterine fibroid lesion are shown in a scenario with fibroid shrinkage, which is common in late follow-up imaging which are performed during more extended period of times after UAE. Image 316 of FIG. 4 displays images of a uterine fibroid lesion prior to UAE wherein there is 100% enhancement and the enhancing volume is 100 cm³. Image 317 of FIG. 4 shows the uterine fibroid lesion after UAE wherein there is 50% enhancement and the enhancing volume is 10 cm³. In this situation, when comparing relative values between A and B, no changes are observable. However, the absolute numeric values indicate significant changes in fibroid enhancement. For instance, the absolute numeric values in images 316 and 317 of FIG. 4 indicate an approximate 40 cm³ reduction in fibroid enhancement. Therefore, while the relative value comparison inaccurately suggests a poor response to UAE the absolute numeric value comparison accurately indicates a significant change in fibroid enhancement. Accordingly, evaluation of uterine fibroid enhancement in absolute numeric values provides an improved assessment of uterine fibroid response to UAE.

It is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments the method and device for three-dimensional quantitative evaluation of uterine fibroids (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. A system for three-dimensional quantitative evaluation, comprising:
a segmentation module configured to perform a three-dimensional segmentation on a contrast-enhanced imaging of a target and to compute the total volume based on the three-dimensional segmentation;
a subtraction module configured to differentiate actual contrast enhancements from background enhancements on the contrast-enhanced imaging and to apply a three-dimensional segmentation mask obtained from the three-dimensional segmentation on the differentiated actual contrast enhancements;
a comparison module configured to define a non-enhanced target portion as a region of interest and to compute contrast statistics for the region of interest in order to determine a normalized threshold value for target enhancement; and
a computation module configured to define the enhanced target as voxels within the three-dimensional mask based on the threshold value and quantify the volume of the enhanced target.

2. The system as recited in claim 1, wherein the subtraction module is configured to:
prepare a subtraction image by subtracting a pre-contrast image from the enhanced contrast image in order to differentiate actual contrast enhancements from background enhancements; and
transfer the three-dimensional segmentation mask to the subtraction image.

3. The system as recited in claim 1, comprising an overlay module that is configured to provide a color map overlay to demonstrate the distribution and intensity of the enhancement.

4. The system as recited in claim 1, wherein the segmentation module is configured to perform the three-dimensional segmentation on a contrast-enhanced imaging of a target comprising a dominant lesion of a uterine fibroid in a living organism.

5. The system as recited in claim 1, wherein the normalized threshold for target enhancement is defined as exceeding the average plus two times the standard deviation value of the region of interest.

6. The system as recited in claim 1 wherein the segmentation module is configured to perform the three-dimensional segmentation on a contrast-enhanced imaging that is prepared by arterial phase magnetic resonance imaging.

7. The system as recited in claim 1 wherein the segmentation module is configured to perform the three-dimensional segmentation on a contrast-enhanced imaging that is prepared by arterial phase cone-beam computed tomography.

8. The system as recited in claim 7, wherein the segmentation module is configured to perform the three-dimensional segmentation on a contrast-enhanced imaging that is performed intra-procedure.

9. A system for three-dimensional quantitative evaluation, comprising:
a processor;
an imaging system comprising arterial-phase magnetic resonance imaging or arterial-phase cone beam computed tomography;
a segmentation module configured to perform a three-dimensional segmentation on a contrast-enhanced imaging of a dominant lesion of the uterine fibroids and to compute the total lesion volume based on the three-dimensional segmentation;
a subtraction module configured to differentiate actual contrast enhancements from background enhancements on the contrast-enhanced imaging and to apply a three-dimensional segmentation mask obtained from the three-dimensional segmentation on the differentiated actual contrast enhancements;
a comparison module configured to define a non-enhanced tissue portion as a region of interest and to compute contrast statistics for the region of interest in order to determine a normalized threshold value for tissue enhancement; and
a computation module configured to define the enhanced fibroid tissue as voxels within the three-dimensional mask based on the threshold value and quantify the volume of the enhanced fibroid tissue.

10. The system as recited in claim 9, wherein the subtraction module is configured to:
prepare a subtraction image by subtracting a pre-contrast image from the enhanced contrast image in order to differentiate actual contrast enhancements from background enhancements; and
transfer the three-dimensional segmentation mask to the subtraction image.

11. The system as recited in claim 9, comprising an overlay module that is configured to provide a color map overlay to demonstrate the distribution and intensity of the enhancement.

12. The system as recited in claim 9, wherein the segmentation module is configured to perform the three-dimensional segmentation performed semi-automatically.

13. The system as recited in claim 9, wherein the normalized threshold for tissue enhancement is defined as exceeding the average plus two times the standard deviation value of the region of interest.

14. The system as recited in claim 9, wherein the imaging system comprises cone beam computed tomography and the segmentation module is configured to perform the three-dimensional segmentation on imaging that is performed intra-procedure.

15. A method for three-dimensional quantitative evaluation of uterine fibroids comprising the steps of:
performing a three-dimensional segmentation on a contrast-enhanced imaging of a target and computing total target volume based on the three-dimensional segmentation;
differentiating actual contrast enhancements from background enhancements on the contrast-enhanced imaging;
applying a three-dimensional segmentation mask obtained from the three-dimensional segmentation on the differentiated actual contrast enhancements;
defining a non-enhanced target portion as a region of interest and computing contrast statistics for the region of interest in order to determine a normalized threshold value for target enhancement; and
defining the enhanced fibroid tissue as voxels within the three-dimensional mask based on the threshold value and quantifying the volume of the enhanced fibroid tissue.

16. (canceled)

17. (canceled)

18. (canceled)

19. (canceled)

20. (canceled)
